(19) United States
(12) Patent Application Publication
(10) Pub. No.: US 2001/0038825 A1
(43) Pub. Date: Nov. 8, 2001

(54) Method and compositions for enhancing pulmonary function and treating pulmonary disorders

(76) Inventor: Robert Duncan Hite, Winston-Salem, NC (US)

Correspondence Address: MYERS BIGEL SIBLEY & SAJOVEC, PO BOX 37428, RALEIGH, NC, 27627, US

(21) Appl. No.: 09/777,155

(22) Filed: Feb. 5, 2001

Related U.S. Application Data
(60) Provisional application No. 60/180,388, filed on Feb. 4, 2000.

(51) Int. Cl. A61K 9/00; A61K 31/66

(52) U.S. Cl. 424/43; 514/120; 514/121

(57) ABSTRACT

Pulmonary function may be increased and pulmonary disorders treated by administering pharmaceutical formulations consisting essentially of phosphatidyl glycerol (PG) and a pharmaceutically acceptable carrier. Pulmonary disorders that may be treated by these methods and formulations include respiratory distress syndrome, asthma, and chronic bronchitis.

### RELATED APPLICATIONS

This application claims the benefit of U.S. Provisional Application No. 60/180,388, filed Feb. 4, 2000, which is incorporated herein by reference in its entirety.

### FEDERAL SUPPORT OF THE INVENTION

This invention was made with support from the United States Government under grant number P01 HL50395 from the National Heart, Lung, and Blood Institute of the National Institutes of Health. The United States Government has certain rights in this invention.

### FIELD OF THE INVENTION

This invention relates to methods and compositions that are useful in enhancing pulmonary function generally, and in treating pulmonary disorders.

### BACKGROUND OF THE INVENTION

Naturally-occurring pulmonary surfactant (PS) lines the alveolar epithelium of mammalian lungs, and is a complex mixture of lipids and proteins. PS promotes the formation of a monolayer at the alveolar air-water interface and, by reducing the surface tension, prevents collapse of the alveolus during expiration. Natural PS contains phospholipids, certain neutral lipids, and proteins, with lipids making up 80% of the composition. The lipid component is composed mainly of dipalmitoyl phosphatidylycholine (dispalmitoyl lecithin), phosphatidyl glycerol, phosphatidylethanolamine, triglycerides cholesterol and cholesterol esters. The protein components of surfactant required for full surfactant properties include a family of apoproteins. The presence of a number of these apoproteins has been shown to enhance the rate of surface-film formation (see, e.g., Whitseft et al., Pediatr. Res., 20, 460 (1986); Avery et al., New Engl. J. Med., 315, 825 (1986)).

Sufficient endogenous pulmonary surfactant is necessary for normal lung function. Certain pulmonary disorders associated with pulmonary inflammation are accordingly associated with deficient or abnormal pulmonary surfactant function. For example, Respiratory Distress Syndrome (RDS) is a debilitating pulmonary disease that is characterized by a decrease in PS at the air/liquid interface of the pulmonary alveolus. The descriptive term “RDS” has been applied to many acute, diffuse, and infiltrative pulmonary lesions of diverse etiologies. Diseases classified generally as Respiratory Distress Syndrome (RDS) range from adult respiratory distress syndromes (ARDS) to a neonatal form, referred to variously as idiopathic RDS or hyaline membrane disease.

The treatment of many inflammatory pulmonary disorders such as RDS has historically been limited to supportive care, including, for example, oxygen administration or mechanical ventilation. More recently, progress has been made in the treatment of RDS, and especially neonatal RDS, by surfactant replacement therapy. (See, e.g., Takahashi, et. al, Biochem. Biophys. Res. Comm., 135, 527-532 (1986); Metcalfe, et al., J. Appl. Physiol. 49, 34-40 (1980)). These surfactant-replacement methods generally involve the intratracheal instillation of various surfactant mixtures in order to replenish pulmonary surfactant content exogenously. These surfactant compositions generally comprise a phospholipid component and an apolipoprotein component, in order to mimic or approximate the components of natural PS.

Although pulmonary installations of mixtures of alveolar surface active phospholipids and surfactant-specific apoproteins appear to be an attractive treatments for RDS, the use of natural apoproteins isolated from animals (e.g., cows and pigs) in surfactant replacement therapy also presents certain disadvantages in that isolating such proteins can be time consuming and expensive. Both human and bovine natural surfactants have been administered into the airways of newborn infants. See, e.g., J. Horbar et al., N. Eng. J. Med. 320, 959 (1989); R. Soll et al., Pediatric Res. 23, 425A (1988). Problems with such natural surfactants include potential contamination with microorganisms and potential sensitization of the patient to proteins therein. Additionally, the inclusion of such natural proteins places the subject at risk of having an immune or allergic reaction to the proteins. Recently, the use of proteins produced by recombinant means in such PS replacement compositions has increased. However, the use of such compositions may also be problematic in that producing such compositions require the time consuming steps of identifying, purifying, cloning and/or synthesizing the desired surfactant proteins.

Despite the foregoing, a number of artificial surfactant formulations that can be used to treat or prevent RDS are known, many of which incorporate an apoprotein or polypeptide component. See. e.g., U.S. Pat. Nos. 4,826,821 and 4,312,860 to Clements; U.S. Pat. No. 5,614,216 to Janoff; U.S. Pat. No. 4,828,844 to Rontgen-Odenthal et al., U.S. Pat. No. 4,973,582 to Yoshida et al, U.S. Pat. No. 4,603,124 to Takei et al;, U.S. Pat. Nos. 5,309,903 and 5,207,220 to Long; and U.S. Pat. No. 5,134,129 to Lichtenberger, the disclosures of which are incorporated herein by reference in their entirety.

U.S. Pat. No. 6,013,619 to Cochrane et al., the disclosure of which is also incorporated herein by reference in its entirety, describes pulmonary surfactant compositions that are prepared using a protein, a polypeptide, an amino acid residue-containing molecule, or another organic molecule having surfactant activity (collectively, “surfactant molecules”), that may include one or more phospholipids, for the treatment of Respiratory Distress Syndrome (RDS). However, as is typical of the other artificial surfactant formulations described above, the composition of Cochrane et al. places emphasis on the importance of the presence of the surfactant molecule (e.g., the protein or polypeptide component) as opposed to the phospholipid component, for the efficacy of the surfactant replacement therapy described therein. For example, the Cochrane et al. reference states that a “polypeptide, when admixed with pharmaceutically acceptable phospholipids, forms a pulmonary surfactant that has greater surfactant activity that the phospholipids alone . . . it should be noted that the eight amino acid residue control peptide p74-81, which does not conform to the teachings of the present invention, did not form a PS having a greater activity than the phospholipid alone, thus indicating that amino acid residue length is a critical feature.” (Emphasis added).

Because of the emphasis on the importance of the inclusion of a surfactant protein or polypeptide in compositions for treating inflammatory lung disorders such as RDS, treatments encompassing or contemplating the preferred and selective replacement of phospholipids alone (i.e., in the absence of the surfactant molecule such as PS apoproteins or polypeptides), and even more particularly the preferred and selective replacement of phosphatidyl glycerol (PG) alone, are not known or described.

### SUMMARY OF THE INVENTION

The present inventor has found that the administration of phosphatidyl glycerol (PG, also referred to herein as the “active compound” or “active agent”) imparts an anti-inflammatory effect to pulmonary tissue (i.e., the tissue of the lungs and airways). Although not wishing to be bound to any specific theory of the invention, the inventor has found that the selective administration of PG to inflamed pulmonary tissue results in a decrease of inflammation as a result of reconstituting improved or normal surfactant activity or decreasing surfactant dysfunction, in spite of the fact that PG alone does not have significant surfactant activity. The present invention thus provides an advantage over current methods of treating inflammatory pulmonary disorders. As stated above, current methods of treating certain inflammatory pulmonary disorders (i.e., RDS) involve the administration or either a natural or synthetic pulmonary surfactant composition (i.e., a composition comprising a surfactant molecule such as an apoprotein, polypeptide or analog thereof, and one or more phospholipids). The obtaining or production of the surfactant apoprotein or polypeptide component of such surfactant compositions is generally time-consuming, difficult and often prohibitively expensive on a large scale (i.e., when generating compositions for the treatment of adults rather than infants). In comparison, PG is easily and inexpensively obtainable through natural or synthetic means. Accordingly, the ability to selectively administer PG, either in the complete absence of surfactant molecules or in a composition with a significantly reduced surfactant molecule component in relation to a selectively high PG component, provides both an economic and administrative advantage over present methods.

In light of the foregoing, a first aspect of the present invention is a method of treating a pulmonary disorder in a subject in need of such treatment, comprising administering to the subject a composition consisting essentially of PG and a pharmaceutically acceptable carrier, in an amount effective to treat the pulmonary disorder.

A second aspect of the present invention is a method of enhancing pulmonary function in a subject in need of or desirous of such enhancement, comprising administering to the subject a composition consisting essentially of PG and a pharmaceutically acceptable carrier, in an amount effective to enhance the pulmonary function of the subject.

A third aspect of the present invention is a pharmaceutical composition useful in the treatment of pulmonary disorders consisting essentially of PG and a pharmaceutically acceptable carrier.

A fourth aspect of the present invention is a composition useful in enhancing pulmonary function consisting essentially of PG and a pharmaceutically acceptable carrier.

A fifth aspect of the present invention is the use of a PG for the preparation of a medicament for treating pulmonary disorders in a subject in need thereof.

A sixth aspect of the present invention is the use of PG as described above for the preparation of a medicament for enhancing pulmonary function in a subject in need or desirous thereof.

The foregoing and other aspects of the present invention are explained in detail in the specification set forth below.

### BRIEF DESCRIPTION OF THE DRAWINGS

FIG. 1 is a graphical illustration of the results of experiments relating to surfactant function. Non-asthmatics (n=10) and asthmatics (n=13) post endobronchial challenge with saline and antigen underwent BAL. Surfactant pellets were isolated from BAL and resuspended in buffered saline (1.0 mg phospholipid/ml) with 5.0 mM CaCl₂. Minimum surface tension (γₘᵢₙ) was measured using a pulsating bubble surfactometer over 10 min at 37° C. The data represent the mean±SEM. The dotted line in the antigen-challenged asthmatic results is used to define “Responders” (≧8.0 mN/m) and “Non-Responders” (<8.0 mN/m). * P<0.05 as compared to non-asthmatic controls and saline-challenged asthmatics.

FIG. 2 graphically illustrates the correlation of eosinophils and protein with surfactant function. The recovery of eosinophils (FIG. 2A) and the concentration of surfactant pellet protein (FIG. 2B) are plotted against minimum surface tension. Responders (closed boxes) are defined as γₘᵢₙ≧8 mN/m after antigen challenge while non-responders (open boxes) are <8 mN/m.

FIG. 3 is a graphical illustration of the LA/SA ratio in asthmatics and non-asthmatics. Large surfactant aggregates (LA) were defined as those in the pellet after ultracentrifugation while small aggregates (SA) were defined as the surfactant remaining in the supernatant. LA and SA was measured by quantitation of lipid phosphorous, and LA/SA ratio was calculated. The data represent the mean±SEM.

FIG. 4 illustrates the correlation of the PC/PG index. The PC/PG index is calculated from the ratio of PC and PG concentrations from antigen-challenged samples and saline-challenged samples, and is plotted against minimum surface tension (FIG. 4A) and LA/SA ratio (FIG. 4B). Responders (closed boxes) are defined as ±ₘᵢₙ≧8 mN/m after antigen challenge while non-responders (open boxes) are <8 mN/m.

FIG. 5 is a graphical illustration of the results of experiments relating to phospholipase hydrolysis of PG. BAL supernatant was incubated with [³H]-PG-labeled surfactant for 4 hours at 37° C. in saline with 5 mM CaCl₂. Hydrolysis represents formation of labeled free fatty acid, which was measured using TLC and scintillation counting. Results are mean±SEM. * P<0.05 as compared to non-asthmatic controls and saline-challenged asthmatics.

FIG. 6 is a graphical illustration of the effect of PG replacement on phospholipase hydrolyzed surfactant.

FIG. 7 is a comparison of the percentage of lung surfactant phospholipids in normal patients and ARDS patients.

FIG. 8 is a comparison of the PC/PG ratio in patients with chronic bronchitis and patients with cystic fibrosis.

FIG. 9 is a graphical illustration of the results of experiments in which endogenous pulmonary surfactant was obtained by lavage of normal (ie., non-asthmatic) subjects, asthmatics 24 hrs after endobronchial instillation of saline or allergen, or from patients with ARDS. Surfactant was washed by centrifugation and analyzed in a bubble surfactometer.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

As provided above, an aspect of the present invention is a method of treating a pulmonary disorder in a subject in need thereof, comprising administering a composition consisting essentially of phosphatidyl glycerol (hereinafter referred to interchangeably as “PG”, the “active compound” or the “active agent”) and a pharmaceutically acceptable carrier. Compositions of the present invention are referred to herein as “PG compositions.”

As used herein, the term “phosphatidyl glycerol” encompasses PG and modifications or derivatives thereof (e.g., 2-oleoyl PG). Phosphatidyl glycerol, as used in the present invention may also be expressed by the following formula:

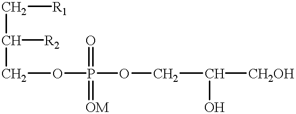

wherein M denotes a cation and R₁ and R₂ indicate straight-chain fatty acid residues. Cations include but are not limited to H⁺, Na⁺, K⁺, NH₄⁺, and Ca²⁺. R₁ and R₂ are preferably straight-chain fatty acid residues having about 14 to about 25 carbon atoms. Examples of such saturated fatty acid residues include but are not limited to myristic acid residues, paimitic acid residues, stearic acid residues, and the like, of which dipalmitoyl phosphatidyl glycerol is preferable. Examples of unsaturated fatty acid residues include but are not limited to oleic acid residues and linoleic acid residues.

In the present invention, phosphatidyl glycerol may be used in its naturally occurring L-configuration, or in an artificial or synthetic DL-configuration, or as a mixture thereof. The naturally occurring L-configuration is preferred.

PG of the present invention may be obtained from naturally occurring sources (i.e., extracted or isolated from plant and animal sources, or even used in its naturally occurring form such as in a plant), or can be synthesized by various known processes. Furthermore, PG is commercially available and may be obtained from known commercial sources. PG may be used in unpurified, partially purified, substantially purified, or even completely purified form.

In one preferred embodiment, PG compositions of the present invention will be essentially or completely free of surfactant molecules or equivalents and analogs thereof. Surfactant molecules may be apoproteins or polypeptides or fragments thereof. Surfactant molecules may also be constituted by alternating groupings of charged and uncharged residues; the residues may be amino acids, modified amino acids, amino acid analogs or derivatives, and the like. Such surfactant molecules are described in U.S. Pat. No. 6,013,619 to Cochrane et al., the disclosure of which is incorporated herein in its entirety.

In an alternative embodiment, the composition of the present invention comprises an artificial surfactant such as those described above, but in which a selectively and preferentially high level of phosphatidyl glycerol is present. Stated another way, a composition of this kind will have a reduced polypeptide or protein component (i.e., surfactant molecule as described above), and a preferentially increased component of PG, as compared to that which is normally used in present methods. In such an embodiment, the phospholipid component of the composition will preferably be PG only (i.e., to the exclusion of other phospholipids), and the concentration of PG will be significantly greater than the concentration of the surfactant component. For example, in such a composition, the weight percentage of PG will consist of 60%, or 70%, or 80%, or 90%, or 95%, or even higher, of the total weight of total active ingredients (i.e., when “total active ingredients” equals surfactant component plus PG). Stated another way, the ratio of PG to surfactant component present in the composition may be 2:1, or 5:1, or 10:1, or 50:1, or 100:1, or 500:1, or 1000:1, or even higher.

The compositions and methods of the present invention are useful in the treatment of pulmonary disorders, and more particularly in the treatment of pulmonary disorders characterized by inflammation of pulmonary tissue. Such disorders include but are not limited to respiratory distress syndrome (RDS) (including adult RDS or ARDS), asthma, bronchitis (both acute and chronic), bronchiectasis, chronic obstructive pulmonary disorder (COPD), pneumonia (including ventilator-associated pneumonia, nosocomial pneumonia, viral pneumonia, bacterial pneumonia, mycobacterial pneumonia, fungal pneumonia, eosinophilic pneumonia, and Pneumocystis carinii pneumonia), tuberculosis, cystic fibrosis (CF), emphysema radiation pneumonitis, inflammation caused by smoking, pulmonary edema, bronchiolitis, pneumoconiosis, sarcoidiosis, silicosis, asbestosis, berylliosis, coal worker's pneumonoconiosis (CWP), byssinosis, interstitial lung diseases (ILD) such as idiopathic pulmonary fibrosis, ILD asociated with collagen vascular disorders, systemic lupus erythematosus, rheumatoid arthritis, ankylosing spondylitis, systemic sclerosis, and pulmonary inflammation that is a result of or is secondary to another disorder such as influenza.

The terms “treating” and “treatment” as used herein refer to any type of treatment that imparts a benefit to a patient afflicted with a pulmonary disorder, including improvement in the condition of the patient (e.g., in one or more symptoms), delay in the progression of the disorder, etc. As used herein, “treatment” is not necessarily meant to imply cure or complete abolition of the disorder. Alternatively stated, and as used herein, “treatment” of a pulmonary disorder refers to methods of inhibiting or slowing the progression of the disorder, reducing the incidence of the disorder, or preventing the disorder. As such, the term “treatment” also includes prophylactic treatment of the subject to prevent the onset of the disorder. The term “prevention of pulmonary disorder” means the elimination or reduction in the incidence or onset of the pulmonary disorder, as compared to that which would occur in the absence of treatment. Alternatively stated, the present methods slow, delay, control, or decrease the likelihood or probability of the pulmonary disorder in the subject, as compared to that which would occur in the absence of treatment.

Subjects that may be treated by the methods of the present invention include those suffering from a pulmonary disorder, and those at risk for developing a pulmonary disorder. At-risk individuals include, but are not limited to, individuals with a family history of pulmonary disorder, individuals who have previously been treated for pulmonary disorders, and individuals presenting any other clinical indicia suggesting that they have an increased likelihood of developing the pulmonary disorder. Alternatively stated, an at-risk individual is any individual who is believed to be at a higher risk than the general population for developing a pulmonary disorder.

The compositions of the present invention may be administered either before or during pulmonary crises. Further, they can also be administered prior to single-lung, double-lung, or heart-lung transplant. In addition, it may be desirable to give the active compound to the subject over a long period as an adjunct to, e.g., the standard therapies for pulmonary disorders.

When used as a pharmaceutical treatment, the compositions of the present invention may be administered either alone or optionally in conjunction with other compounds or compositions that are used in the treatment of pulmonary disorders. For example, if a subject is being treated for a pulmonary disorder caused by a bacterial infection, then a composition of the present invention may be administered in conjunction with another compound or treatment used to treat the bacterial infection, such as an antibiotic. Examples of such compounds, referred to herein as “supplemental compounds,” or “supplemental compositions,” include, but are not limited to, antibiotics, anti-cytokines, anti-asthma drugs, antiphospholipases (e.g., inhibitors of phospolipase), vasodilators (e.g., adenosine, β-adrenergic agonists or antagonists, β-adrenergic blockers, α-adrenergic blockers, diuretics, smooth muscle vasodilators, nitrates, and angiotensin-converting enzyme inhibitors), and compounds found to be useful in the treatment of cystic fibrosis, such as pyrazinoylguanidine sodium channel blockers (e.g., amiloride, benzamil, phenamil) and P2Y₂ receptor agonist (e.g., UTP, U₂P₄, etc.).

The co-administration of supplemental compounds or compositions can be performed before, after, or during the administration of the PG composition. The supplemental compounds or compositions may optionally be administered concurrently. As used herein, the word “concurrently” means sufficiently close in time to produce a combined effect (that is, concurrently may be simultaneously, or it may be two or more events occurring within a short time period before or after each other). Simultaneous administration may be carried out at the same point in time but at different anatomic sites or using different routes of administration.

In addition to being useful in methods of treating lung disorders (i.e., where the PG composition is a pharmaceutical composition), the PG compositions and methods of the present invention are useful in enhancing pulmonary or airway function in subjects generally. In this aspect of the invention, the subject is not necessarily suffering from a pulmonary disorder, but rather may be a subject who is generally physically healthy, but desires improvement in pulmonary function (i.e., easier breathing). Subjects may desire the administration of the PG compositions in this context, for example, because of incidental exposure to cigarette smoke (primary or second-hand), environmental toxins or air pollution or smog; because of a decrease in pulmonary function due to normal aging processes; because of the desire for improved cardiovascular fitness, and the like. In this sense, the compositions of the present invention are administered not as a prescribed pharmaceutical (i.e., a drug), but rather as a general health-improving tonic. By “enhance” is meant that pulmonary function (e.g., respiration) occurs at an improved level, as compared to pulmonary function occurring with the lack of administration of the PG compositions of the present invention.

The present invention is primarily concerned with the treatment of human subjects, but the invention may also be carried out on animal subjects, particularly mammalian subjects such as primates, mice, rats, dogs, cats, livestock, and horses for veterinary purposes, and for drug screening and drug development purposes. Human subjects include infants, neonates, juveniles, adolescents, and adults.

Compositions of the present invention consist essentially of PG and a pharmaceutically or physiologically acceptable carrier. The terms “pharmaceutically acceptable” or “physiologically acceptable,” as used herein, mean that the carrier is suitable for administration to a subject to achieve the treatments or enhancement/tonic administrations described herein, is compatible with any other ingredients in the formulation, and is not unduly deleterious to the subject in light of the severity of the disorder (if present) and the necessity or desirability of the treatment. Any pharmaceutically acceptable carrier may be employed in the present invention, such as sterile saline solution, sterile water, etc.

The compositions described herein may be formulated for administration in a pharmaceutical carrier in accordance with known techniques. See, e.g., Remington, The Science And Practice of Pharmacy 9th Ed. (A. R. Gennaro, ed., Mack Publishing Co., Easton, Pa., 1995). In the manufacture of a composition according to the invention, the PG is typically admixed with, inter alia, the pharmaceutically acceptable carrier. The carrier may be a solid or a liquid, or both, and is preferably formulated with the compound as a unit-dose formulation, which may contain from 0.01% or 0.5% to 95% or 99% by weight of the active compound.

The pharmaceutical formulation may optionally include one or more accessory ingredients. Thus, in addition to PG, the composition may contain additives such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use.

Further, the present invention provides PG compositions in liposomal form. The technology for forming liposomal suspensions is well known in the art. Due to its water-solubility, PG is incorporated into lipid vesicles by being substantially entrained within the hydrophilic center or core of the liposomes. The lipid layer employed may be of any conventional composition and may either contain cholesterol or be cholesterol-free. Liposomes that are produced by these techniques may be reduced in size, through the use of standard sonication and homogenization techniques. Of course, the liposomal formulations containing the compounds disclosed herein or salts thereof may be lyophilized to produce a lyophilizate. The lyophilizate may be reconstituted with a pharmaceutically acceptable carrier, such as water, to regenerate a liposomal suspension.

The compositions are preferably administered directly into the lungs of the subject by any suitable means, but preferably by intratracheal instillation, and more preferably bolus intratracheal instillation. An alternative preferred method of administration is via inhalation of respirable aerosol particles comprising the composition. Pulmonary lavage is another alternative route of administration. Accordingly, the composition may be administered, as appropriate to the dosage form, by endotracheal tube, by bronchoscope, by cannula, by aerosol administration, or by nebulization of a suspension or dust of the composition into a gas to be inspired. Compositions of the present invention may also be delivered to the lung in an aerosolized form using the pulmonary drug delivery systems set forth in U.S. Pat. No. 5,874,064 to Edwards et al. and U.S. Pat. No. 5,934,273 to Andersson et al., the disclosures of which are incorporated herein in their entireties.

In one embodiment of the invention, the PG composition is administered by administering an aerosol suspension of respirable particles comprised of the PG composition, which the subject inhales. The PG composition can be aerosolized in a variety of forms, such as, but not limited to, dry powder inhalants, metered dose inhalants, or liquid/liquid suspensions. The respirable particles may be liquid or solid.

Solid or liquid particulate forms of the PG compositions prepared for practicing the present invention should include particles of respirable size: that is, particles of a size sufficiently small to pass through the mouth and larynx upon inhalation and into the bronchi and alveoli of the lungs. In general, particles ranging from about 1 to 10 microns in size are within the respirable range. Particles of non-respirable size that are included in the aerosol tend to be deposited in the throat and swallowed, and the quantity of non-respirable particles in the aerosol is preferably minimized. The particulate pharmaceutical composition may optionally be combined with a carrier to aid in dispersion or transport. A suitable carrier such as a sugar (i.e., lactose, sucrose, trehalose, mannitol) may be blended with the active compound or compounds in any suitable ratio (e.g., a 1 to 1 ratio by weight).

Aerosols of liquid particles comprising the PG compositions may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active compound (i.e., PG) into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, antioxidants, flavoring agents, volatile oils, buffering agents, and surfactants.

Aerosols of solid particles comprising the PG composition may likewise be produced with any solid particulate medicament aerosol generator. Aerosol generators for administering solid particulate medicaments to a subject produce particles that are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders that may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

A second type of illustrative aerosol generator comprises a metered dose inhaler. Metered dose inhalers are pressurized aerosol dispensers, typically containing a suspension or solution formulation of the active ingredient in a liquified propellant. During use, these devices discharge the formulation through a valve adapted to deliver a metered volume, typically from 10 to 200 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

Any propellant may be used in carrying out the present invention, including both chlorofluorocarbon-containing propellants and non-chlorofluorocarbon-containing propellants. Thus, fluorocarbon aerosol propellants that may be employed in carrying out the present invention including fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to: CF₃—CHF—CF₂H; CF₃—CH₂—CF₂H; CF₃—CHF—CF₃; CF₃—CH₂—CF₃; CF₃—CHCl—CF₂Cl; CF₃—CHCl—CF₃; cy-C(CF₂)₃—CHCl; CF₃—CHCl—CH₂Cl; CF₃—CHF—CF₂Cl; CF₃—CHCl—CFHCl; CF₃—CFCl—CFHCl; CF₃—CF₂—CF₂H; CF₃—CF₂—CH₃; CF₂H—CF₂—CFH₂; CF₃—CF₂—CFH₂; CF₃—CF₂—CH₂Cl; CF₂H—CF₂—CH₃; CF₂H—CF₂—CH₂Cl; CF₃—CF₂—CF₂—CH₃; CF₃—CF₂—CF₂—CF₂H; CF₃—CHF—CHF—CF₃; CF₃—O—CF₃; CF₃—O—CF₂H; CF₂H—H—O—CF₂H; CF₂H—O—CFH₂; CF₃—O—CH₃; CF₃—O—CF₂—CF₂H; CF₃—O—CF₂—O—CF₃; cy-CF₂—CF₂—O—CF₂—; cy-CHF—CF₂—O—CF₂—; cy-CH₂—CF₂—O—CF₂—; cy-CF₂—O—CF₂—O—CF₂—; CF₃—O—CF₂—Br; CF₂H—O—CF₂—Br; and mixtures thereof, where “cy” denotes a cyclic compound in which the end terminal covalent bonds of the structures shown are the same so that the end terminal groups are covalently bonded together. Particularly preferred are hydrofluoroalkanes such as 1,1,1,2-tetrafluoroethane and heptafluoropropane. A stabilizer such as a fluoropolymer may optionally be included in formulations of fluorocarbon propellants, such as described in U.S. Pat. No. 5,376,359 to Johnson.

Compositions containing respirable dry particles of micronized PG may be prepared by grinding dry PG with, e.g., a mortar and pestle or other appropriate grinding device, and then passing the micronized composition through a 400 mesh screen to break up or separate out large agglomerates.

The aerosol, whether formed from solid or liquid particles, may be produced by the aerosol generator at a rate of from about 10 to 150 liters per minute. Aerosols containing greater amounts of medicament may be administered more rapidly. Typically, each aerosol may be delivered to the patient for a period from about 30 seconds to about 20 minutes, with a delivery period of about five to ten minutes being preferred.

Regardless of the route of administration of the active compounds or formulations of the invention, the therapeutically effective dosage of any one active compound, the use of which is in the scope of present invention, will vary somewhat from compound to compound, and patient to patient, and will depend upon factors such as the age, weight and condition of the patient, and the route of delivery. Such dosages can be determined in accordance with routine pharmacological procedures known to those skilled in the art. For example, as a general proposition, for an adult human a dosage from 1 mg PG/kg body weight to 50 mg PG/kg body weight per dose, is effective, with a range of from about 1 mg PG/kg body weight to 50 mg PG/kg body weight being preferred, and with all weights being calculated based upon the weight of the active compound. In the case of repetitive treatments (e.g., over the course of a week), an administration range of from about 10 mg/kg body weight per week up to about 1000 mg/kg body weight per week is effective. Of course, in newly born infants, the dose will be commensurately lower; in such infants, one or two administrations are generally sufficient. For adults, the composition is preferably administered to produce a PO₂ within the normal range (see, e.g., Hallman et al, J. Clinical Investigation, 70, 673-682 (1982)). When administration of the PG composition is via inhalation, the dosage of PG will also vary depending on the condition being treated and the state of the subject, but generally may be an amount sufficient to achieve dissolved concentrations of PG on the airway surfaces of the subject of from about 10⁻⁹ to about 10⁻¹ moles/liter, and more preferably from about 10⁻⁶ to about 10⁻⁴ moles/liter.

The treatment regimens of the invention may vary from individual to individual, depending on the severity of the disorder, the symptoms present, and other relevant variables. Thus, single or multiple doses may be administered to an individual. Depending upon the solubility of the particular composition of PG administered to a subject, the daily dose may be divided among one or several unit dose administrations. The duration of the treatment is usually once per day for a period of time that will vary by subject, but will generally last until the pulmonary disorder is essentially controlled or the desired level of pulmonary enhancement achieved. Lower doses given less frequently can be used prophylactically to prevent or reduce the incidence of recurrence of the pulmonary disorder, or to maintain healthy pulmonary function. The doses of the PG compositions may be provided as one or several prepackaged units.

The following Examples are provided to illustrate the present invention, and should not be construed as limiting thereof.

### EXAMPLE 1

### Patient Selection

Subjects with atopic asthma (n=13) and normal, non-smoking, non-asthmatic subjects without a history of chronic illness or allergy (n=10) were studied. Each asthmatic met criteria of reversible airflow obstruction demonstrated by an increase in FEV1 of ≧15% following beta agonist inhalation, or PC₂₀ methacholine <8 mg/ml, and episodic symptoms of including cough, dyspnea and wheezing. Subjects with significant cigarette smoking exposure (<5 pack years) were excluded. Anti-inflammatory medications including inhaled and systemic corticosteroids, cromones, and leukotriene modifiers were withheld for at least 6 weeks before study initiation. Inhaled beta-agonists (except salmeterol) were withheld for six hours, and salmeterol and theophylline were withheld for 24 hours prior to each study.

### EXAMPLE 2

### Antigen Challenge

To determine the dose used for endobronchial challenge, inhaled antigen challenges were performed in each asthmatic subject using cat dander (Felis domesticus), dust mite (Dermatophagoides farinae), short ragweed (Greer Laboratories; Lenoir, N.C.) or standardized cat hair (ALK Laboratories; Wallingford, Conn.) based on results of prior skin testing. Antigens were administered via a continuous LC Jet+ nebulizer (PARI Respiratory Equipment; Richmond, Va.) over two minutes of tidal breathing. The FEV1 was measured at five-minute intervals, and the antigen concentration increased every 15 minutes until a 20% drop in FEV1 or the highest concentration (1:10) was achieved. Subjects were monitored for a minimum of 10 hours following antigen challenge, including determination of LAR. All subjects experienced a LAR defined as a drop in FEV1 of at least 15% which persisted for >30 minutes (nadir in FEV1 occurred 6.4±0.4 hours after challenge). Five ml of a 1:10 dilution of the concentration resulting in a 20% fall in FEV1 was used for the segmental challenge.

### EXAMPLE 3

### Endobronchial Antigen/Saline Challenge

Bronchoscopy for challenge was performed on day 1 with segmental challenge in either the lingula or right middle lobe using 5 cc of antigen solution described above (and the bronchoscope channel flushed with 5 cc saline), and 10 cc of saline on the contralateral side. Bronchoscopy was repeated 24 hours later (day 2), and bilateral BAL's were performed.

### EXAMPLE 4

### Bronchoalveolar Lavage (BAL)

Bronchoalveolar lavage (BAL) was performed 24 hours following endobronchial application of antigen to a single segment of one lung and saline to a single segment of the opposite lung. For comparison to normal controls, BAL was obtained from non-asthmatics that did not undergo antigen stimulation.

Fiberoptic bronchoscopy was performed using nebulized and topical Xylocaine with benzodiazepine sedation. After a single inhalation (0.131 mg) of isoproterenol or albuterol (0.09 mg) via MDI was administered (to asthmatic subjects only), the bronchoscope was introduced transnasally into the lower airways. For asthmatic subjects, BAL was performed using three 50 ml aliquots (150 ml total volume) of sterile saline, and BAL fluid (BALF) was kept on ice until processed (approximately 30 minutes). The BAL data from non-asthmatic subjects are from separate studies, which used a larger volume of saline (four 50 ml aliquots=200 ml total volume).

### EXAMPLE 5

### Cell Isolation

The total BAL was measured for volume, and centrifuged at 200×g for 15 minutes. The supernatant was removed and further processed for isolation of surfactant (below). The resulting pellet was washed three times with PBS and resuspended to a final concentration of 1×10⁷ cells/ml in PBS containing 0.1% gelatin. Total cell count and viability were determined by hemacytometer and trypan blue exclusion. Differential cell counts were determined using Leukostat stain.

### EXAMPLE 6

### Isolation of Surfactant

BAL supernatant (cell free) was centrifuged at 45,000×g for 1 hour. Aliquots of the resulting supernatant were stored at −70□° C. Large surfactant aggregates (LA) were defined as those in the pellet after ultracentrifugation while small aggregates (SA) were defined as the surfactant remaining in the supernatant. See Veldhuizen, R. A., et al., Biochem J 295:141-147 (1993). The surfactant pellet was washed three times with normal saline (pH 7.4), and resuspended in a known volume of normal saline. Aliquots were stored at −70° C. The phospholipid content of LA and SA was measured by quantitation of lipid phosphorous using the methods of Bartlett (J Biol Chem 234:466468 (1959)). “Total” surfactant recovered represents the sum of LA and SA.

### EXAMPLE 7

### Surfactant Function

A pulsating bubble surfactometer (Electronetics; Amherst N.Y.) was used to measure surface tension lowering activity. See Enhorning, G., J Appl Physiol 43, 198-203 (1977). Surfactant from the LA fraction (40 μl) at a final concentration of 1.0 mg phospholipid/ml in buffered saline with 5 mM CaCl₂ was used, and analyzed at 20 cycles/min for 10 min at 37□° C. The results are reported as γₘᵢₙ, which represents the minimum surface tension achieved over the 10 minute period.

### EXAMPLE 8

### Protein

The protein content of the BAL supernatant and the surfactant pellet was measured using the BCA method (Pierce; Rockford, Ill.), with BSA as standards.

### EXAMPLE 9

### Phospholipid Composition

The phospholipid fraction of samples was separated using lipid extraction via the methods of Bligh and Dyer (Can J Biochem Physiol 37:911-917 (1959)). After extraction, the organic phase is isolated, dried under N₂, resuspended in chloroform and analyzed for phospholipid concentration. Phospholipid composition of LA was determined by separation of individual phospholipids with high performance liquid chromatography (HPLC) using a Kromasil column (Keystone; Bellefonte, Pa.) and a dual solvent system with initially 100% Mobile Phase A (CHCl₃:MeOH:NH₄OH; 80:19.5:0.5) ramped to a maximum of 25% Mobile Phase B (MeOH:H₂O:NH₄OH; 80:19.5:0.5). Quantitation of individual phospholipid peaks from the HPLC eluent was performed with an evaporative light scatter detector (SEDERE; Alfortville, France). Retention times and binomial response characteristics of individual phospholipids and lysophospholipids were defined using commercially available standards. This sensitive method measures individual phospholipids in the range of 0.1 to 4.0 nmoles, and requires a minimum surfactant sample size of 12 nmoles. To calculate absolute phospholipid concentrations, a fixed concentration of phosphatidylbutanol (PB) was added to a known concentration of sample after lipid extraction to serve as an internal standard.

### EXAMPLE 10

### PG Hydrolysis

Supernatant from each BAL (475 μl) was incubated with Survantao (Ross Laboratories; St. Louis, Mo.), which was labeled with [³H]-oleate-PG (0.026 nmoles lipid/μl, 0.004 μCi/μl) using repetitive vortexing. The [³H]-PG was prepared by TLC purification of PG from membranes of [³H]-oleate-labeled Escherichia coli using previously reported methods. See Kramer, R. M. et al., Methods Enzymol 197:373-381 (1991); Shinozaki, K. et al., Biochemistry 38:1669-1675 (1999).

The phospholipid concentration of the final mixture (500 μl) was 1.0 mg/ml (583.4 nmoles of phospholipid/sample) with the [³H]-labeled PG contributing only 0.13 nmoles (0.02 μCi/sample). The reaction mixture was supplemented with CaCl₂ (5.0 mM), and incubated at 37° C. for 4 hours. Lipid extraction was used to stop hydrolysis. The phospholipid and free fatty acid fractions from the organic phase of the lipid extraction were measured using previously reported methods. See Hite, R. D., et al., Am J Physiol 19:L740-L747 (1998). Hydrolysis is expressed as the percent of the total radioactivity in the free fatty acid fraction.

### EXAMPLE 11

### Statistics

Data represent the mean±SEM. Statistical significance was determined using paired and unpaired Student's t tests and Spearman rank correlation.

### EXAMPLE 12

### Surfactant Function

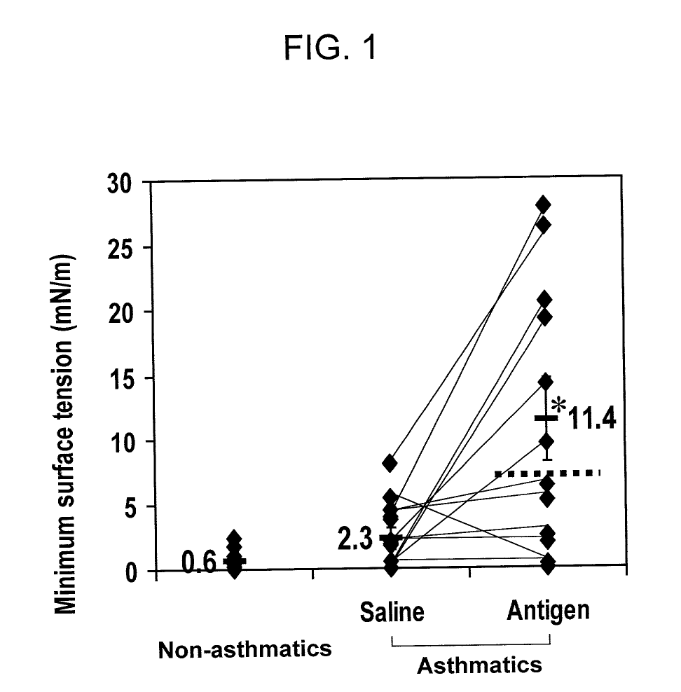

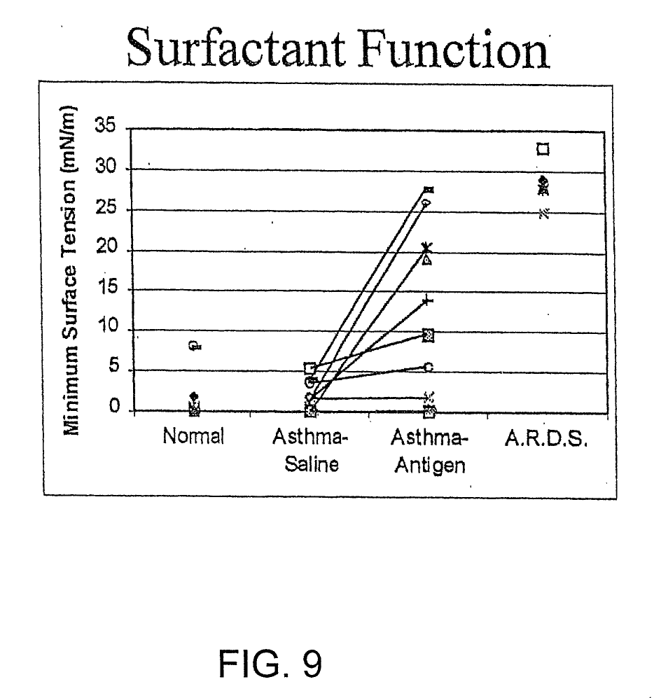

Minimum surface tension (γₘᵢₙ) of the surfactant pellets from non-asthmatic subjects and asthmatics (saline- and antigen-challenged samples from each) are shown in FIG. 1. No significant difference in γₘᵢₙ was seen between the saline-challenged asthmatic samples and the non-asthmatics. However, the antigen-challenged asthmatic samples demonstrated significantly higher γₘᵢₙ than the corresponding saline-challenged samples (11.4±3.2 vs. 2.3±0.8 mN/m, p<0.05). For comparison, surfactant data from ARDS patients is included in FIG. 9. As shown therein, in some patients, degrees of abnormality approached those of the ARDS patients, even in mild asthmatics.

There was a broad range of response in the asthmatics with approximately half (6 of 13) demonstrating significantly increased γₘᵢₙ. To further investigate the key differences, which might identify important mechanisms for surfactant dysfunction, subjects were separated into “responders” and “non-responders” for subsequent analyses. These groups are differentiated by the γₘᵢₙ value of the antigen-challenged sample. A ±ₘᵢₙ cutoff of 8.0 mN/m (dotted line in FIG. 1) was chosen since as it approximates the upper 95% confidence limit in the saline-challenged samples (7.7 mN/m), and was the point within the antigen-challenged samples which delineated subjects with a significant increase in γₘᵢₙ.

For Tables 1, 2 and 3, the data is separated into the following groups: non-asthmatic samples, saline-challenged asthmatic samples and antigen-challenged asthmatic samples. Both the saline-challenged and antigen-challenged data are further separated into non-responders, responders and total (non-responders and responders combined).

TABLE 1
Profile of BAL cells.

| | | Asthmatics | | | Asthmatics | | |
|---|---|---|---|---|---|---|---|
| | | Saline-Challenged | | | Antigen-Challenged | | |
| | Non | | Non | | | Non | |
| | Asthmatics | Total | Responders | Responders | Total | Responders | Responders |
| | (n = 10) | (n = 13) | (n = 7) | (n = 6) | (n = 13) | (n = 7) | (n = 6) |
| CELLULAR RESPONSE | | | | | | | |
| Total Cells (× 10⁴/ml) | 6.5 ± 1.1 | 37.6 ± 6.2^{a} | 51.8 ± 8.7 | 28.1 ± 6.2 | 138.2 ± 46.1^{a,b} | 56.2 ± 26.5 | 192.8 ± 67.7^{b} |
| Eosinophils (× 10⁴/ml) | <0.1 | 1.3 ± 0.7 | 1.2 ± 0.5 | 1.3 ± 1.2 | 77.9 ± 30.9^{a,b} | 23.5 ± 19.3 | 114.2 ± 45.4^{b} |
| Neutrophils (× 10⁴/ml) | <0.1 | 11.4 ± 4.7^{a} | 20.6 ± 7.8 | 5.2 ± 4.8 | 10.7 ± 3.7^{a} | 9.9 ± 5.1 | 11.3 ± 5.5 |
| Lymphocytes (× 10⁴/ml) | 0.4 ± 0.1 | 2.4 ± 0.5^{a} | 3.1 ± 0.5 | 1.9 ± 0.8 | 14.3 ± 16.7 | 3.5 ± 1.7 | 21.5 ± 10.4 |
| Alveolar Macs (× 10⁴/ml) | 5.9 ± 1.0 | 22.4 ± 2.2^{a} | 26.8 ± 4.1 | 19.6 ± 2.1 | 35.0 ± 11.0^{a} | 19.1 ± 7.0 | 45.6 ± 17.0 |

^{a}p < 0.05 comparison of asthmatics (saline or antigen-challenged) to non-asthmatics
^{b}p < 0.05 comparison of paired asthmatic samples (antigen-challenge v. saline-challenge)

TABLE 2
Protein concentrations and surfactant recovery

| | | Asthmatics | | | Asthmatics | | |
|---|---|---|---|---|---|---|---|
| | | Saline-Challenged | | | Antigen-Challenged | | |
| | Non- | | Non | | | Non | |
| | Asthmatics | Total | Responders | Responders | Total | Responders | Responders |
| | (n = 10) | (n = 13) | (n = 7) | (n = 6) | (n = 13) | (n = 7) | (n = 6) |
| PROTEIN | | | | | | | |
| BAL Supernatant | 63.7 ± 10.2 | 221.7 ± 116.3 | 130.0 ± 22.7 | 305.8 ± 215.1 | 1384.9 ± 697.3 | 198.8 ± 87.3 | 2280.5 ± 1191.0 |
| (μ/ml) | | | | | | | |
| Surfactant Pellet | 239.2 ± 8.3 | 201.5 ± 7.7^{a} | 208.4 ± 9.1 | 197.5 ± 10.8 | 334.9 ± 47.0^{b} | 272.1 ± 37.5 | 376.8 ± 72.1^{b} |
| (μg/mg Phospholipid) | | | | | | | |

SURFACTANT RECOVERY AND AGGREGATES

| | | | | | | | |
|---|---|---|---|---|---|---|---|
| Total (μg/ml) | 24.5 ± 3.1 | 32.3 ± 4.3 | 36.7 ± 6.9 | 28.7 ± 5.0 | 25.4 ± 5.2 | 26.8 ± 4.1 | 25.9 ± 6.1 |
| Large Aggregates-LA (μg/ml) | 20.6 ± 3.2 | 25.3 ± 2.8 | 28.9 ± 4.7 | 22.9 ± 3.6 | 16.4 ± 2.5^{b} | 20.5 ± 4.5 | 13.6 ± 2.7 |
| Small Aggregates-SA (μg/ml) | 3.8 ± 0.9 | 6.3 ± 1.2 | 7.7 ± 2.4 | 4.9 ± 1.2 | 8.7 ± 1.4 | 4.8 ± 0.6 | 11.7 ± 1.0^{b,c} |
| LA/SA Ratio | 6.6 ± 1.6 | 4.8 ± 0.4 | 4.2 ± 0.6 | 5.3 ± 0.4 | 2.6 ± 0.6^{a,b} | 4.4 ± 0.7 | 1.3 ± 0.3^{b,c} |

^{a}p < 0.05 comparison of asthmatics (saline or antigen-challenged) to non-asthmatics
^{b}p < 0.05 comparison of paired asthmatic samples (antigen-challenge v. saline-challenge)
^{c}p < 0.05 comparison of responder to non-responders (saline- or antigen-challenged)

TABLE 3
Surfactant phospholipid composition.

| | | Asthmatics | | | Asthmatics | | |
|---|---|---|---|---|---|---|---|
| | | Saline-Challenged | | | Antigen-Challenged | | |
| | Non- | | Non | | | Non | |
| | Asthmatics | Total | Responders | Responders | Total | Responders | Responders |
| | (n = 10) | (n = 13) | (n = 7) | (n = 6) | (n = 13) | (n = 7) | (n = 6) |
| PHOSPHOLIPID COMPOSITION | | | | | | | |
| Phosphatidylcholine (μg/ml) | 14.1 ± 1.2 | 20.5 ± 2.3^{a} | 23.2 ± 3.1 | 18.3 ± 2.7 | 14.0 ± 1.9^{b} | 15.4 ± 3.5 | 12.9 ± 1.3 |
| Phosphatidyiglycerol (μg/ml) | 2.2 ± 0.2 | 3.4 ± 0.5 | 3.6 ± 0.5 | 3.3 ± 0.6 | 2.5 ± 0.4 | 3.4 ± 0.5 | 1.7 ± 0.2^{b,c} |
| Phosphatidylethanolamine (μg/ml) | 0.8 ± 0.1 | 0.8 ± 0.2 | 1.0 ± 0.2 | 0.8 ± 0.2 | 0.8 ± 0.1 | 0.9 ± 0.1 | 0.8 ± 0.2 |
| Phosphatidylinositol (μg/ml) | 0.5 ± 0.1 | 0.9 ± 0.2 | 0.7 ± 0.1 | 1.0 ± 0.3 | 0.6 ± 0.1 | 0.8 ± 0.1 | 0.4 ± 0.1^{c} |
| Sphingomyelin (μg/ml) | 0.1 ± 0.1 | 0.4 ± 0.3 | 0.3 ± 0.1 | 0.5 ± 0.4 | 0.2 ± 0.1 | 0.1 ± 0.1 | 0.2 ± 0.2 |
| PC/PG | 6.8 ± 0.5 | 6.4 ± 0.6 | 6.8 ± 0.5 | 6.2 ± 0.8 | 6.5 ± 0.9 | 4.8 ± 0.9 | 7.8 ± 1.0^{c} |
| PC/PG Index | | | | | 1.0 ± 0.14 | 0.68 ± 0.09 | 1.32 ± 0.12^{c} |
| (Antigen v. Saline) | | | | | | | |

^{a}p < 0.05 comparison of asthmatics (saline or antigen-challenged) to non-asthmatics
^{b}p < 0.05 comparison of paired asthmatic samples (antigen-challenge v. saline-challenge)
^{c}p < 0.05 comparison of responder to non-responders (saline- or antigen-challenged)

The characteristics of baseline asthma and asthma exacerbation were analyzed by comparison of non-asthmatic samples with all saline-challenged samples and with all antigen-challenged samples, respectively. To analyze the characteristics of antigen challenge, all antigen-challenged samples were challenged to all saline-challenged samples. The changes characteristic of surfactant dysfunction after antigen challenge were analyzed by two methods: (1) comparison of the antigen-challenged responders and non-responders to their corresponding saline-challenged samples and (2) comparison of the antigen-challenged samples of responders to the antigen-challenged samples of non-responders.

### EXAMPLE 13

### BAL Recovery

There was no difference in the volume of BAL recovered between all saline-challenged asthmatic samples and all antigen-challenged asthmatic samples (54.8% vs. 52.8%). Similarly, there were no differences with BAL recovery in the responder and non-responder subgroups. The recovery of BAL was significantly greater in the non-asthmatics on comparison to either the saline-challenged asthmatic samples (73.6% vs. 54.8%, p<0.001) or the antigen-challenged asthmatic samples (73.6% vs. 52.8%, p<0.001). The differences seen in non-asthmatics likely reflect the increased airway collapse during aspiration of BAL from asthmatic airways, and the larger volume of BAL used in the non-asthmatic lavages.

### EXAMPLE 14

### BAL Cellular Response

As shown in Table 1, total BAL cells (10⁴ cells/ml) were increased in antigen-challenged asthmatic samples on comparison with both the saline-challenged asthmatic samples (138.2 vs. 37.6, p<0.05) and the non-asthmatics (138.2 vs. 6.5, p<0.03). Total cells were also increased in the saline-challenged samples versus the non-asthmatic samples (37.6 vs. 6.5, p<0.001). Within the asthmatics, total cells only increased significantly within the responder subset (192.8 vs. 28.1, p<0.05). The inflammatory response to antigen challenge was likely the most significant factor in the differences between antigen-challenged samples to saline-challenged samples. Similarly, the inflammation from antigen challenge and chronic asthma are important factors in the differences between non-asthmatics and asthmatics, but other factors including the bronchoscopic manipulation of the airways 24 hours before the BAL during instillation in the asthmatics and the different BAL volumes were also likely influential.

On analysis of specific cell types, antigen-challenged samples (vs. saline-challenged samples) had significantly increased eosinophils (77.9 vs. 1.3, p<0.03). Analysis of responder and non-responder subsets, revealed the responders had significant increases in eosinophils (114.2 vs. 1.3, p<0.05), while the responders did not. Although total cells and eosinophils tended to increase on comparison of responders to non-responders, the differences were not significant (p<0.1).

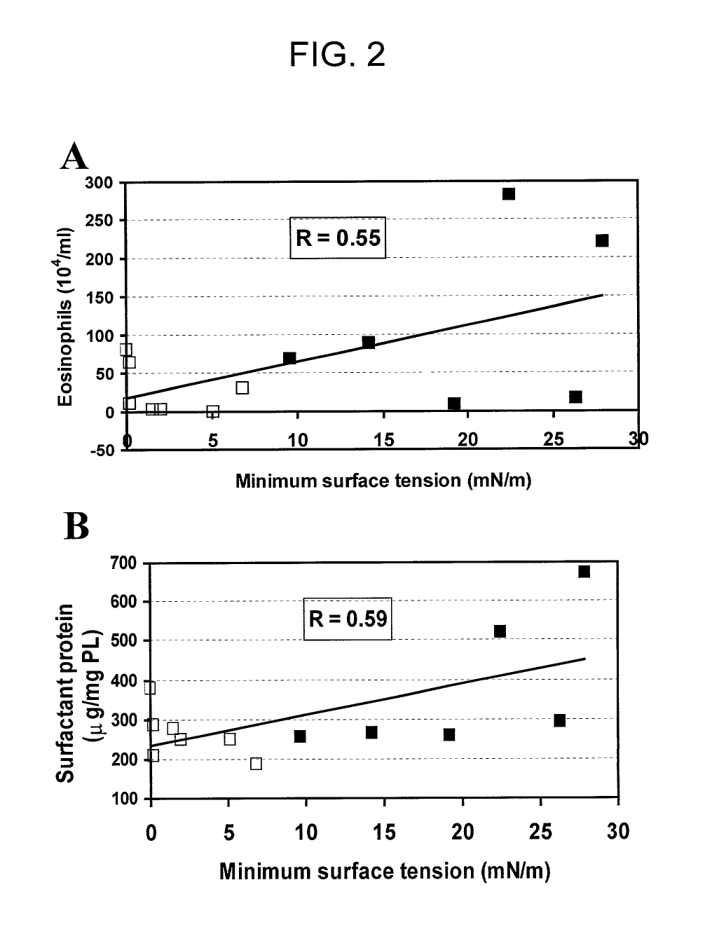

To examine the predictive value of the cellular changes on surfactant dysfunction, we performed correlation analysis between total eosinophil count and ±ₘᵢₙ (FIG. 2A). Eosinophils were selected for the following reasons: (1) the only cell type to increase significantly in response to antigen challenge in our data and (2) previously implicated as being important in surfactant dysfunction. Although the correlation was statistically significant (R=0.55, p<0.05), it was weak with many patients having significant changes in ±ₘᵢₙ but minor changes in eosinophils, and other patients with significant changes in eosinophils without significant change in γₘᵢₙ.

### EXAMPLE 15

### Bal Protein

The two protein concentrations in Table 2 represent the protein content of (a) the supernatant after removal of cells and LA and (b) the surfactant pellet, or LA. This distinction was principally made to identify the protein concentrations present during our surfactant functional analysis, because BAL supernatant was not utilized in those analyses. The concentration of the surfactant pellet has been calculated as μg of protein per mg of phospholipid since function studies were performed at a fixed phospholipid concentration of 1 mg/ml.

BAL supernatant protein in the total antigen-challenged group was highly variable, and not significantly increased on comparison to the total saline-challenged group. In contrast, increased protein was seen in the surfactant pellet on comparison between the antigen-challenged samples and the saline-challenged samples (334.9 vs. 201.5, p<0.01). This difference was only seen in the responders (376.8 vs. 197.5, p<0.05), but not the non-responders. However, no significant difference was seen on direct comparison between antigen-challenged responders and antigen-challenged non-responders. Interestingly, pellet protein was significantly decreased in the saline-challenged asthmatics versus the non-asthmatics (239.2 vs. 201.5, p<0.005).

The importance of the protein increases after antigen challenge on surfactant dysfunction was examined using correlation analysis between surfactant pellet protein and γₘᵢₙ (FIG. 2B). Similar to the results seen with eosinophils, there is a significant correlation (R=0.59, p<0.03), which is slightly stronger than for eosinophils, but does not reliably predict surfactant dysfunction. A similar degree of correlation was seen when comparing the BAL supernatant protein with γₘᵢₙ (data not shown).

### EXAMPLE 16

### Surfactant Phospholipid Recovery

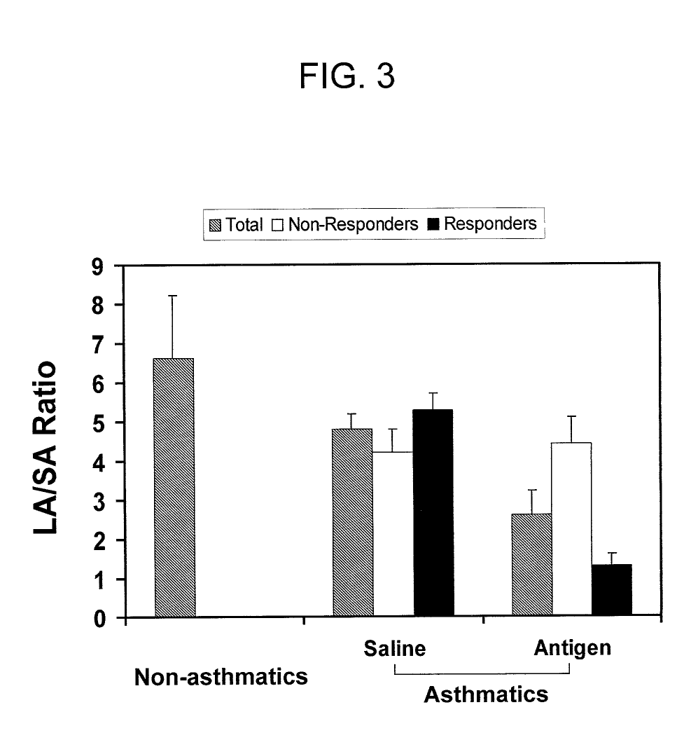

The differences in LA, SA and the LA/SA ratio in all groups as measured by the phospholipid content are provided in Table 2 and highlighted in FIG. 3. There were no significant differences in total surfactant phospholipid recovery between any of the groups. A significant decrease in LA/SA ratio was seen after antigen challenge in the total group of asthmatics (2.6 vs. 4.8, p=0.005). On subset analysis, the decreased LA/SA ratio was seen in the responder group (1.3 vs. 5.3, p<0.001), but not the non-responders. A significantly lower LA/SA ratio was also seen on comparison of antigen-challenged responders to antigen-challenged non-responders (1.3 vs. 4.4, p<0.005).

The decreased LA/SA ratio in the total asthmatic group primarily resulted from a decrease in LA, which was significantly reduced after antigen (16.4 vs. 25.3, p<0.05), and approached significance in the responders (13.6 vs. 22.9, p=0.066). However, increased SA contributed to the differences in LA/SA ratio on comparisons between responders and non-responders. After antigen challenge, SA increased significantly in responders (11.7 vs. 4.9, p=0.001), but not in the non-responders. Similarly, SA was significantly greater in the antigen-challenged responders than the antigen-challenged non-responders (11.7 vs. 4.8, p<0.001). Correlation between the decrease in LA/SA ratio with the increase in γₘᵢₙ was high (R=−0.77, p<0.005), suggesting this marker accurately predicts surfactant dysfunction.

### EXAMPLE 17

### Phospholipid Composition

Table 3 details an analysis of phospholipid composition (μg/ml) from the surfactant pellet using HPLC/ELSD. A significant decrease in PG was seen after antigen challenge in the responders (1.7 vs. 3.3, p<0.05), but was not seen in the non-responders or the total asthmatic group. The PG recovered was also significantly different on direct comparison of the antigen-challenged responders to the antigen-challenged non-responders (1.7 vs. 3.4, p<0.02). These differences in PG were statistically significant, despite the small sample sizes and an unpaired analysis, which suggested PG changes might be more valid predictors of surfactant dysfunction.

In addition to the changes in PG, PC decreased on comparison of the total antigen-challenged group to the total saline-challenged group (14.0 vs. 20.5, p<0.05). Although PC decreased in both the responder and non-responder subsets, neither group reached statistical significance and was not different on direct comparison of responders versus non-responders. Interestingly, a decrease in phosphatidylinositol (PI), an anionic phospholipid similar to PG, was also seen in the comparison of antigen-challenged responders versus antigen-challenged non-responders (0.4 vs. 0.8, p<0.02).

To further analyze the relative importance of the decrease in PG, the PG recovery relative to the recovery of PC (PC/PG) was examined, since the interaction of these two phospholipids is important for the surface activity of in vitro surfactant monolayers. See Dhand, R., et al., Lung 177: 127-138 (1999). A significant difference was seen on comparison of the antigen-challenged responders to the antigen-challenged non-responders. The responders revealed significantly increased PC/PG (7.8 vs. 4.8, p<0.05) which reflected primarily the decrease in PG, since PC content in both groups decreased. In addition, we calculated the relative change in PC/PG between antigen-challenged and saline-challenged samples (calculated by dividing PC/PG of antigen by PC/PG of saline sample) for each individual asthmatic creating a PC/PG index. Using this index, the difference between non-responders and responders was highly significant (1.32 vs. 0.68, p=0.001).

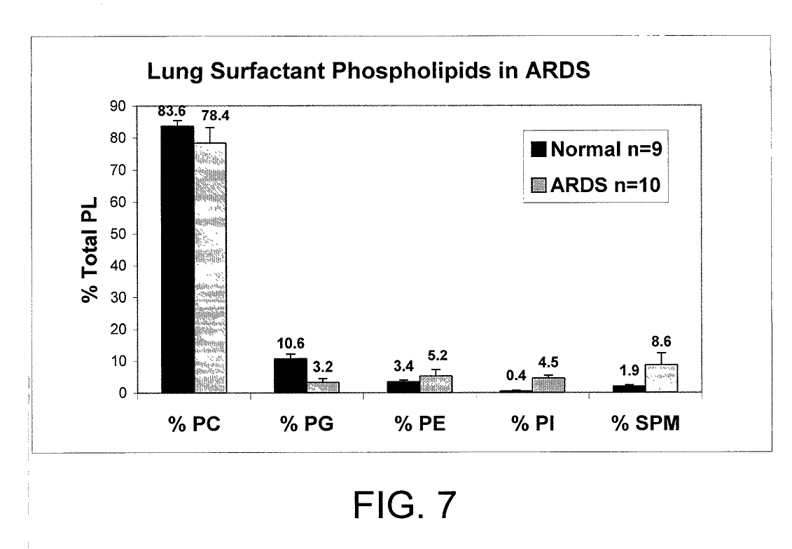

FIG. 7 is a comparison of the percentage of lung surfactant phospholipids in normal patients and ARDS patients. The data for this figure was obtained using the foregoing methods.

### EXAMPLE 18

### Correlation of PC/PG Index

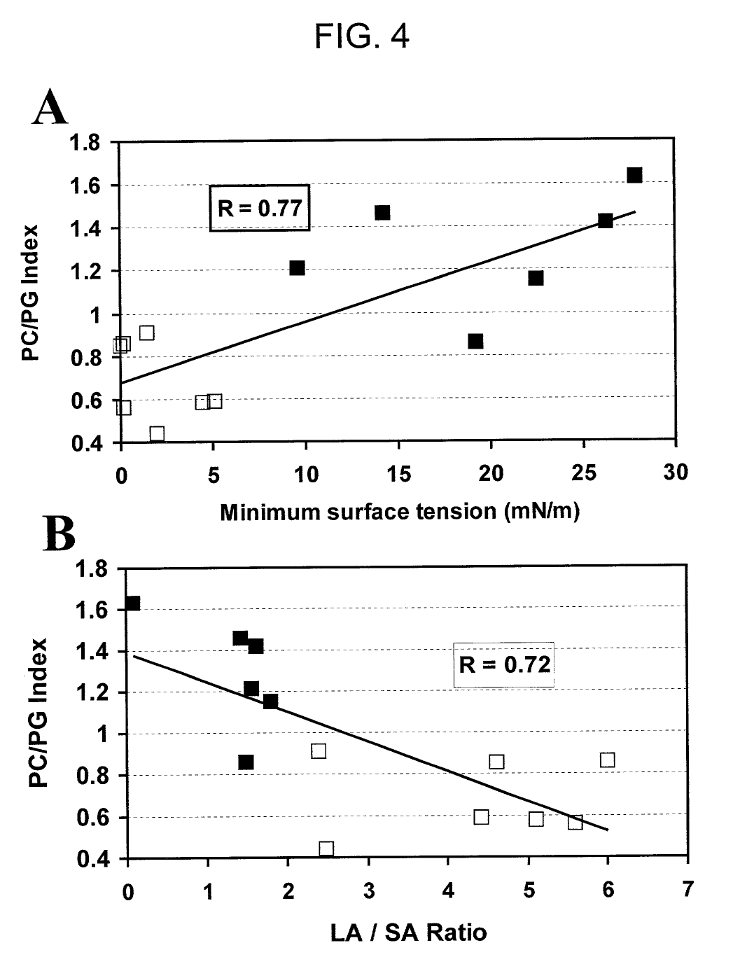

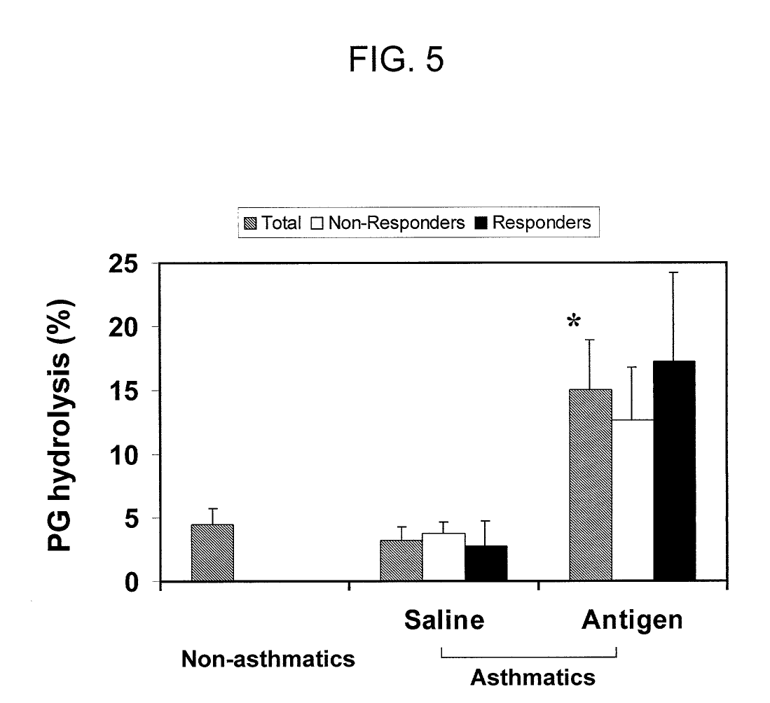

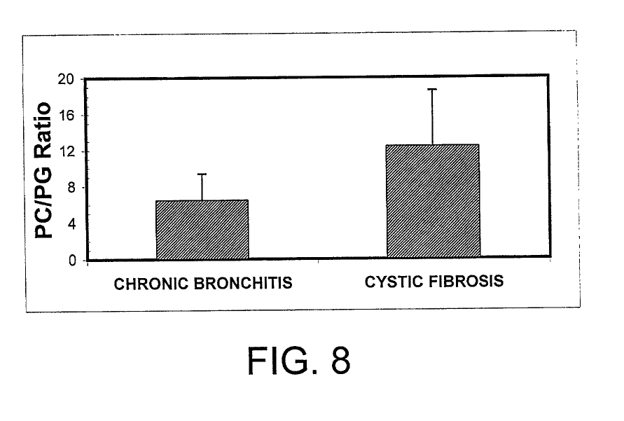

FIG. 4 highlights the strong correlation between the PC/PG index and γₘᵢₙ (R=0.76, p<0.005). Correlation analysis of the PC/PG index with the LA/SA ratio (FIG. 5), an indirect marker of dysfunctional surfactant, similarly demonstrates a very strong correlation (R=−0.72, p<0.005). These correlations suggest that the PC/PG index, a specific surfactant-associated change, predicts surfactant dysfunction after antigen challenge better than any of the other endpoints studied. For comparison, FIG. 8 shows a comparison of the PC/PG ratio in patients with chronic bronchitis and patients with cystic fibrosis.

### EXAMPLE 19

### Phospholipase Activity

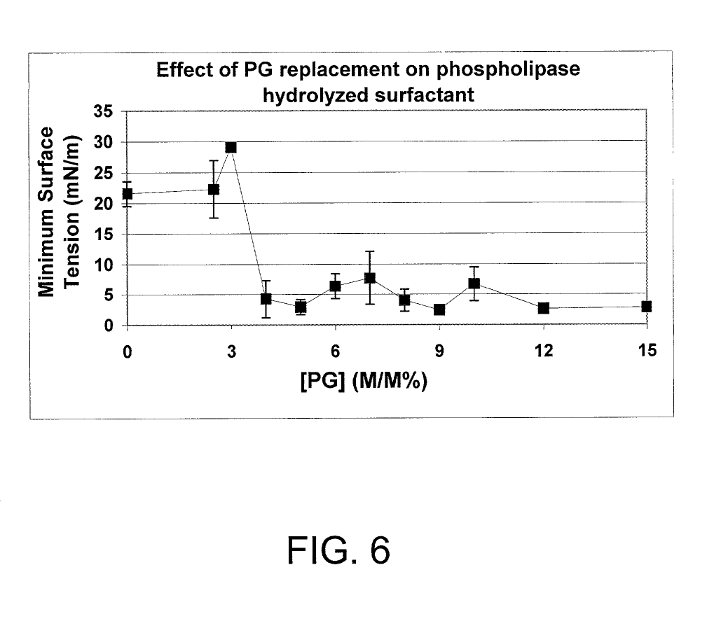

The phospholipase A₂ (PLA₂) activity of the BAL supernatant from the above samples was measured by hydrolysis of [³H]-PG-labeled exogenous surfactant (FIG. 5). Hydrolysis of PG is significantly greater in the antigen-challenged samples on comparison to the saline-challenged samples (15.0 vs. 3.2, p<0.01) and the non-asthmatics (15.0 vs. 4.5, p<0.05). This increased PLA₂ activity after antigen challenge approached but did not achieve significance on subset analysis of both responders and non-responders (p<0.1) due to smaller number of subjects in the subsets. No difference was detected on direct comparison of antigen-challenged responders to antigen-challenged non-responders. FIG. 6 graphically illustrates the effect of PG replacement on phospholipase hydrolyzed surfactant.

The foregoing illustrates the decrease in the PG content of surfactant after antigen challenge that correlates more closely with surfactant dysfunction and conversion of LA to SA than do the increases in airway eosinophils and BAL protein. The foregoing also confirms the importance of surfactant abnormalities in pulmonary disorders such as asthma and ARDS. The data shows that an absolute reduction in PG and its subsequent contribution to the PC/PG index correlates tightly with key surfactant functional abnormalities.

The foregoing is illustrative of the present invention and is not to be construed as limiting thereof. The invention is defined by the following claims, with equivalents of the claims to be included therein.

That which is claimed is:

1. A method of treating a pulmonary disorder in a subject in need thereof, comprising administering to the subject a composition consisting essentially of phosphatidyl glycerol (PG) and a pharmaceutically acceptable carrier in an amount effective to treat the pulmonary disorder.

2. The method according to claim 1 in which the PG is naturally occurring.

3. The method according to claim 1 in which the PG is synthetic.

4. The method according to claim 1 in which the pulmonary disorder is selected from the group consisting of asthma, respiratory distress syndrome (RDS), cystic fibrosis (CF), bronchitis, and pneumonia.

5. The method according to claim 1 in which the pulmonary disorder is RDS.

6. The method according to claim 1 in which the pulmonary disorder is asthma.

7. The method according to claim 1 in which the subject is an adult human.

8. The method according to claim 1 in which the composition is administered by intratracheal instillation.

9. The method according to claim 1 in which the composition is administered by inhalation administration.

10. The method according to claim 9 in which the composition is administered with a nebulizer.

11. The method according to claim 1 in which the phosphatidyl glycerol is administered in a dosage of from about 10 mg/kg to about 50 mg/kg per dose.

12. The method according to claim 1 in which the composition is administered concurrently with a supplemental composition useful for treating the pulmonary disorder.

13. The method according to claim 12 in which the supplemental composition comprises a compound selected from the group consisting of antibiotics, anticytokines, anti-asthma drugs, and amiloride.

14. A method of enhancing pulmonary function in a subject in need thereof, comprising administering to the subject a composition consisting essentially of phosphatidyl glycerol (PG) and a pharmaceutically acceptable carrier in an amount effective to enhance pulmonary function in the subject.

15. The method according to claim 14 in which the composition is administered by inhalation administration.

16. The method according to claim 15 in which the composition is administered with a nebulizer.

17. The method according to claim 14 in which the PG is naturally occurring.

18. The method according to claim 14 in which the PG is synthetic.

19. A pharmaceutical composition useful in treating pulmonary disorders consisting essentially of phosphatidyl glycerol and a pharmaceutically acceptable carrier.

20. The pharmaceutical composition according to claim 19 wherein the composition is a liposomal composition.

21. The pharmaceutical composition according to claim 19 wherein the composition is aerosolized.

21. The pharmaceutical composition according to claim 19 wherein the composition is in liquid form.

22. A pharmaceutical composition useful in enhancing pulmonary function consisting essentially of phosphatidyl glycerol and a pharmaceutically acceptable carrier.

23. The pharmaceutical composition according to claim 19 wherein the composition is a liposomal composition.

24. The pharmaceutical composition according to claim 19 wherein the composition is aerosolized.

25. The pharmaceutical composition according to claim 19 wherein the composition is in liquid form.
